(19) United States
(12) Patent Application Publication
(10) Pub. No.: US 2010/0030199 A1
(43) Pub. Date: Feb. 4, 2010

(54) Configurations And Methods For Power Generation In LNG Regasification Terminals

(75) Inventor: John Mak, Santa Ana, CA (US)

Correspondence Address: FISH & ASSOCIATES, PC;ROBERT D. FISH, 2603 Main Street, Suite 1000, Irvine, CA, 92614-6232, US

(73) Assignee: FLUOR TECHNOLOGIES CORPORATION, Aliso Viejo, CA (US)

(21) Appl. No.: 11/917,387

(22) PCT Filed: Jul. 17, 2006
(86) PCT No.: PCT/US06/27798

§ 371 Date: Sep. 15, 2009

Related U.S. Application Data
(60) Provisional application No. 60/700,021, filed on Jul. 15, 2005.

(51) Int. Cl. A61K 9/22; A61M 5/142

(52) U.S. Cl. 604/892.1; 604/151

(57) ABSTRACT

Disclosed are embodiments of a fluid delivery device (300) that combine both anionic electrokinetic and cationic electrokinetic concepts. In one illustrative embodiment, the fluid delivery device (300) may include an electro-osmotic pump (302) having an anion exchange membrane (350) and a cation exchange membrane (351) in the same device.

### RELATED APPLICATIONS

This application claims priority to U.S. Provisional Patent Application No. 60/700,021, filed Jul. 15, 2005, and titled “Dual Membrane Electro-Osmotic Fluid Delivery Device,” which is incorporated herein by reference.

### BRIEF DESCRIPTION OF THE DRAWINGS

Understanding that drawings depict only certain embodiments of the disclosure and are therefore not to be considered limiting of its scope, the embodiments will be described and explained with additional specificity and detail through the use of the accompanying drawings in which:

FIG. 1 is a block diagram of one embodiment of an anionic electrokinetic-based fluid delivery device including an electro-osmotic engine.

FIG. 2 is a block diagram of one embodiment of a cationic electrokinetic-based fluid delivery device including an electro-osmotic engine.

FIG. 3 is a block diagram of one embodiment of a dual membrane electro-osmotic fluid delivery device.

FIG. 4 is a block diagram of another embodiment of a dual membrane electro-osmotic fluid delivery device having more than one fluid reservoir.

FIG. 5A is a block diagram of one embodiment of an implantable dual membrane electro-osmotic fluid delivery device.

FIG. 5B is a block diagram of one embodiment of a dual membrane electro-osmotic fluid delivery device that may be disposed external to a patient.

FIG. 6 is a block diagram of another embodiment of a dual membrane electro-osmotic fluid delivery device that may be disposed external to a patient.

### DETAILED DESCRIPTION

In the following description, numerous specific details are provided for a thorough understanding of specific embodiments. However, those skilled in the art will recognize that embodiments can be practiced without one or more of the specific details, or with other methods, components, materials, etc. In some cases, well-known structures, materials, or operations are not shown or described in detail. Furthermore, the described features, structures, or characteristics may be combined in any suitable manner in a variety of alternative embodiments.

Disclosed are embodiments of systems, methods, and apparatus relating to fluid delivery devices. The term “fluid” is meant to include a liquid, gel, paste, or other semi-solid state or flowable material that is capable of being delivered out of a reservoir. In some embodiments, these fluid delivery devices are capable of delivering a small amount of a beneficial agent over a period of time. The term “beneficial agent” is meant to include, but is not limited to, any therapeutic agent or drug, medicament, vitamin, lubricant, chemical agent or solution that can be administered to produce a desired, usually beneficial effect.

In some embodiments, the fluid delivery devices may be implantable in a patient. The term “patient” is to be construed broadly to include humans and other animals. In other embodiments, the fluid delivery devices may be disposed outside of the body of a patient, while remaining in fluid communication with the body surface or internal to the body of the patient, such as through a needle, catheter and the like. In yet other embodiments, the fluid delivery devices may be used in non-medical applications, such as the delivery of fragrances, disinfectants, etc.

Exemplary fluid delivery devices having components that may be used in connection with embodiments of the systems, devices, and methods disclosed herein can be found in U.S. Patent Application Publication No. 2003/0205582 titled “Fluid Delivery Device Having an Electrochemical Pump with an Anionic Exchange Membrane and Associated Method,” U.S. Pat. No. 5,744,014 titled “Storage Stable Electrolytic Gas Generator for Fluid Dispensing Applications,” and U.S. Pat. No. 5,707,499 titled “Storage-stable, Fluid Dispensing Device Using a Hydrogen Gas Generator.” Each of the foregoing references are hereby incorporated by reference.

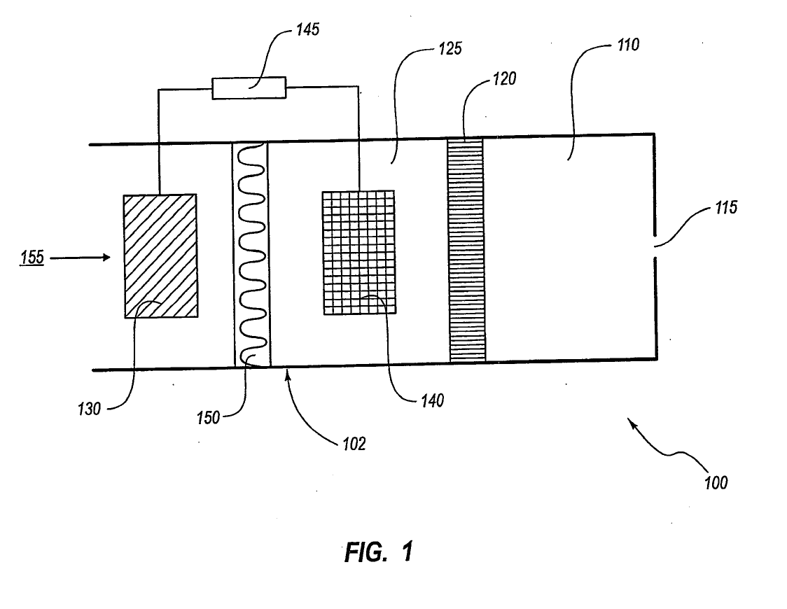

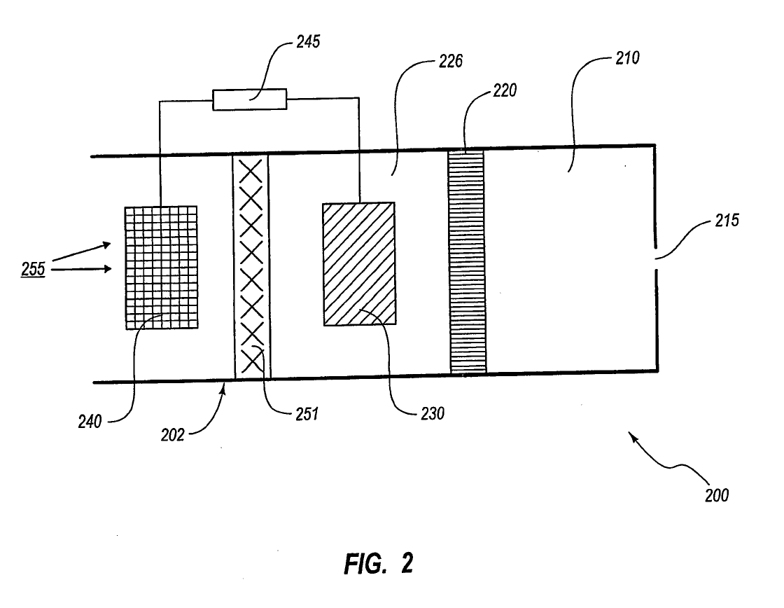

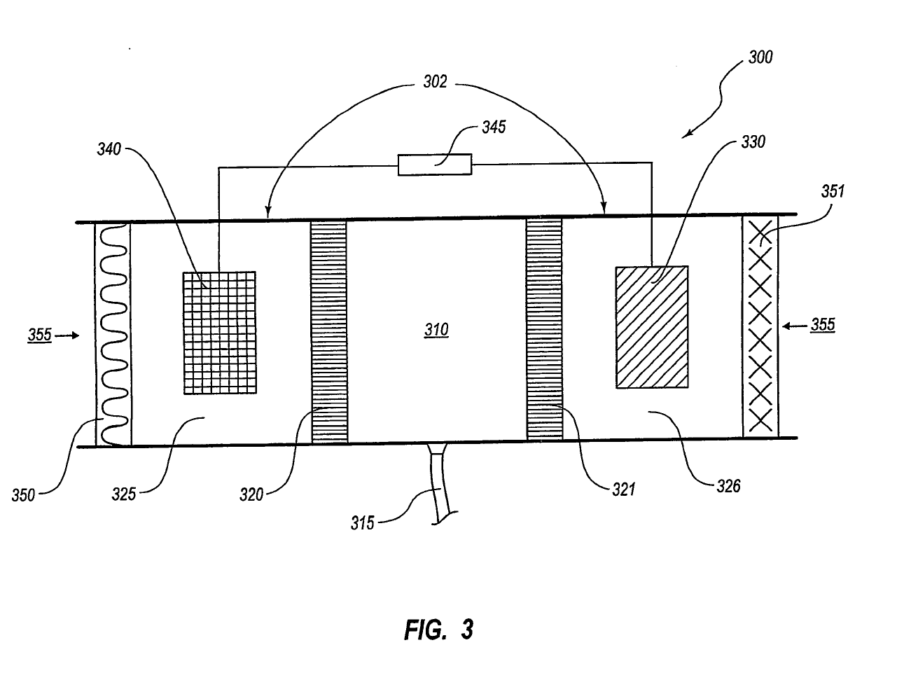

Further details of specific illustrative embodiments will now be described with reference to the accompanying drawings. While FIG. 1 and FIG. 2 represent systems using a single type of ion exchange membrane, the components, methods and materials used may also be used with the embodiments described in conjunction with FIG. 3 through FIG. 6. FIG. 1 depicts a fluid delivery device 100 including an electrochemical pump 102 or engine. Fluid delivery device 100 comprises a fluid reservoir 110. The fluid reservoir 110 may comprise a chamber having fixed, rigid or semi-rigid walls, or alternatively may comprise a bag, bladder, bellows or the like.

The fluid reservoir 110 may house a beneficial agent such as a drug. Fluid reservoir 110 includes a port 115 or orifice, through which the fluid stored in fluid reservoir 110 may be dispensed. It should be understood that, in some embodiments, port 115 may be in fluid communication with a catheter, tube, or other fluid delivery component. A piston 120 or other displaceable member may be positioned to slide within or otherwise apply pressure to reservoir 110 so as to be capable of driving the fluid stored in reservoir 110 through port 115. Alternative displaceable members include, but are not limited to, a bellows, a bladder, a diaphragm, a plunger, and combinations thereof.

The electrochemical engine or pump 102 is configured to provide a force against the piston 120 or other displaceable member to facilitate dispensing fluid out of the fluid reservoir port 115. In one embodiment, such as the embodiment of FIG. 1, the electrochemical pump 102 is an electro-osmotic pump capable of transporting water. An electro-osmotic pump may move fluid by the application of an electric field through an electro-osmotic mechanism.

The electrochemical pump 102 includes a first electrode 130 which may comprise a cathode, and a second electrode 140 which may comprise an anode. Electrodes 130 and 140 may be connected via circuit element 145. Circuit element 145 may comprise a resistor or series of resistors. In some embodiments, the resistor(s) may be replaceable or adjustable so as to vary the rate at which the electrochemical device operates. For example, an adjustable resistor may control the fluid delivery rate. In other embodiments, the circuit element 145 may comprise a switch or other electrical component including a component which merely completes the circuit between electrodes 130 and 140.

An ion exchange membrane 150 is positioned between the two electrodes 130, 140 to provide ionic communication therebetween. In the embodiment of FIG. 1, the ion exchange membrane 150 comprises an anion exchange membrane 150. The anion exchange membrane 150 allows the transport of anions from adjacent the cathode 130 to a driving chamber 125, which houses the anode 140. Consequently, the use of the anion exchange membrane 150 in the electrochemical pump 102 depicted in FIG. 1 means the device 100 is an anionic electrokinetic (“ANEK”) system. However, it should be appreciated that the principles set forth herein are applicable to both ANEK systems and cationic electrokinetic (“CATEK”) systems, as will be discussed in conjunction with FIG. 2.

In the system of FIG. 1, the cathode 130 is disposed outside of the driving chamber 125, and may be exposed to body fluid 155 and/or a saline solution. The cathode 130 may comprise a metal chloride cathode 130, such as silver chloride. Alternative metal chloride cathodes which may be used include high oxidation state cupric, ruthenium, platinum, palladium, iridium or gold chlorides. Furthermore, reducible cathodes such as MnO₂ or AgO may also be used.

According to another embodiment, the cathode 130 may be an oxygen-reducing cathode. Oxygen-reducing cathodes may be enzymatic, such as bilirubin oxidase, laccase, and cytochrome c oxidase. Furthermore, traditional fuel cell cathodes, such as silver, platinum or metal oxide loaded on a conductive carbon substrate, may be used as an oxygen reducing cathode. Porphyrin-based oxygen reducing cathodes may also be used.

When a silver chloride cathode 130 is used during operation of the electrochemical pump 102, silver chloride is reduced to metallic silver, thereby releasing chloride ions into the solution around the electrode according to the equation:

2AgCl+2e⁻→2Ag+2Cl⁻ (1)

The chloride ions generated in the reduction of silver chloride and the chloride ions that are present in the body fluid 155 of a patient migrate through the anion exchange membrane 150 under the influence of the electric field generated by the electrochemical pump 102. These anions move through membrane 150 toward the anode 140 that may be disposed within driving chamber 125 adjacent piston 120.

In the embodiment of FIG. 1, the anode 140 is disposed inside of driving chamber 125. The anode 140 may comprise zinc or other metal or metal containing electrode. Alternatively, enzymatic anodes such as a glucose-oxidizing anode or a lactate-oxidizing anode may be used. Furthermore, traditional metal, polymer, carbon and ceramic based electrocatalysts may be used as well.

The system of FIG. 1, illustrates the use of a zinc anode 130. When the electrochemical pump 102 is activated, zinc is oxidized and dissolved according to the equation:

Zn→Zn²⁺+2e⁻ (2)

The combination of zinc ions thus formed and the chloride ions that pass though the anion exchange membrane 150 form soluble zinc chloride according to the equation:

Zn²⁺+2Cl⁻→ZnCl₂ (3)

During the transport of chloride ions across the anion exchange membrane 150, a sheath of water molecules is entrained with the chloride ions such that, at the opposite side of the membrane 150, an additional amount of water is generated. This electrokinetic water transport is known in the art as electro-osmotic transport. The water molecules transported into the driving chamber 125 generate pressure which can be used to drive piston 120 (or other displaceable member) and deliver the fluid within reservoir 110.

The steady buildup of ions in the driving chamber 125 due to the transport of chloride ions and the cations produced at the anode 140 induces further water transport through an osmotic effect. For instance, if a zinc anode were used as the anode 140, an equilibrium concentration of zinc chloride may be established in the driving chamber 125 after a period of operation resulting in water transport via the osmotic effect. The anion exchange membrane 150 may allow some back diffusion of zinc chloride from the driving chamber 125 toward the cathode 130. Thus, a steady-state flux of water transport into the driving chamber 125 is established by combined electro-osmotic and osmotic effects.

FIG. 2 depicts another embodiment of a fluid delivery device 200 having one ion exchange membrane. Like fluid delivery device 100, fluid delivery device 200 includes a fluid reservoir 210 with a port 215 and a displaceable member such as a piston 220 to facilitate dispensing fluid out of fluid reservoir 210. The fluid delivery device 200 also includes an electrochemical pump 202 which, in one embodiment, may be an electro-osmotic pump comprising a first electrode 230 coupled to a second electrode 240 via circuit 245. However, in the embodiment of FIG. 2, a cation exchange membrane 251 may be positioned between electrodes 230 and 240. Electrode 240 may be an anode that is located outside of driving chamber 226. Electrode 230 may be a cathode that is disposed inside driving chamber 226. The fluid delivery device 200 is, therefore, a CATEK system.

In a CATEK system, the redox reactions may be the same as the ANEK system, however, the electrode positions are different. Once the electrochemical pump 202 is activated in a CATEK system, cations, such as Zn²⁺ generated through oxidation of anode 240 and Na⁺, present in body fluid 255, migrate under the influence of the electric field through the cation exchange membrane 251 towards the cathode 230 in the driving chamber 226. The combination of osmotic and electro-osmotic effects provides pressure in the driving chamber 226 to dispense the fluid from fluid reservoir 210.

FIG. 3 depicts one embodiment of a dual membrane fluid delivery device 300. Like the fluid delivery devices described in conjunction with FIG. 1 and FIG. 2, the dual membrane fluid delivery device 300 may include a fluid reservoir 310 to house a fluid such as a beneficial agent. The fluid delivery device 300 also includes an electrochemical pump 302, which may be an electro-osmotic pump comprising a first electrode 330, such as a cathode, coupled to a second electrode 340, such as an anode, via circuit element 345. The fluid delivery device 300 may include a catheter 315 or similar fluid delivery component to direct the delivery of the beneficial agent from the fluid reservoir 310.

The dual membrane fluid delivery device 300 combines both ANEK and CATEK systems into a single device. For instance, the anode 340 may be disposed inside first driving chamber 325. Driving chamber 325 may be defined by the walls of the device in combination with a first piston 320 (or other displaceable member) and an anion exchange membrane 350. The cathode 330 may be disposed inside a second driving chamber 326 that may be defined by the device walls in combination with a second piston 321 (or alternative displaceable member) and a cation exchange membrane 351.

Once the electrochemical pump 302 is activated, anions, such as Cl⁻ from body fluid 355, migrate under the influence of the electric field through the anion exchange membrane 350 into the first driving chamber 325. As was explained previously, water is transported across the anion exchange membrane 350 through combined electro-osmotic and osmotic effects, thereby generating pressure within first driving chamber 325 which can be used to drive first piston 320 and delivery fluid within reservoir 310.

Simultaneously, cations, such as Na⁺ from body fluid 355, migrate under the influence of the electric field through the cation exchange membrane 351 towards the cathode 330 in the driving chamber 326. Water transport across the cation exchange membrane 351 is accomplished through combined electro-osmotic and osmotic effects. Pressure is thereby generated within second driving chamber 326, which can be used to drive second piston 321 and deliver fluid from within reservoir 310.

The embodiment depicted in FIG. 3 provides for pressure to be exerted from either side of fluid reservoir 310, by first and second driving chambers 325, 326 to controllably expel fluid via catheter 315 or other orifice. While FIG. 3 is not drawn to scale, having a single electrochemical pump 302 that can be used to drive two pistons 320, 321 decreases the ratio of the electro-osmotic engine volume to volume of fluid to be dispensed compared to those shown in FIG. 1 and FIG. 2. Furthermore, the embodiment of FIG. 3 provides for an increase in the electro-osmotic flux using the same two electrodes that are used in single membrane systems such as those shown in FIG. 1 and FIG. 2.

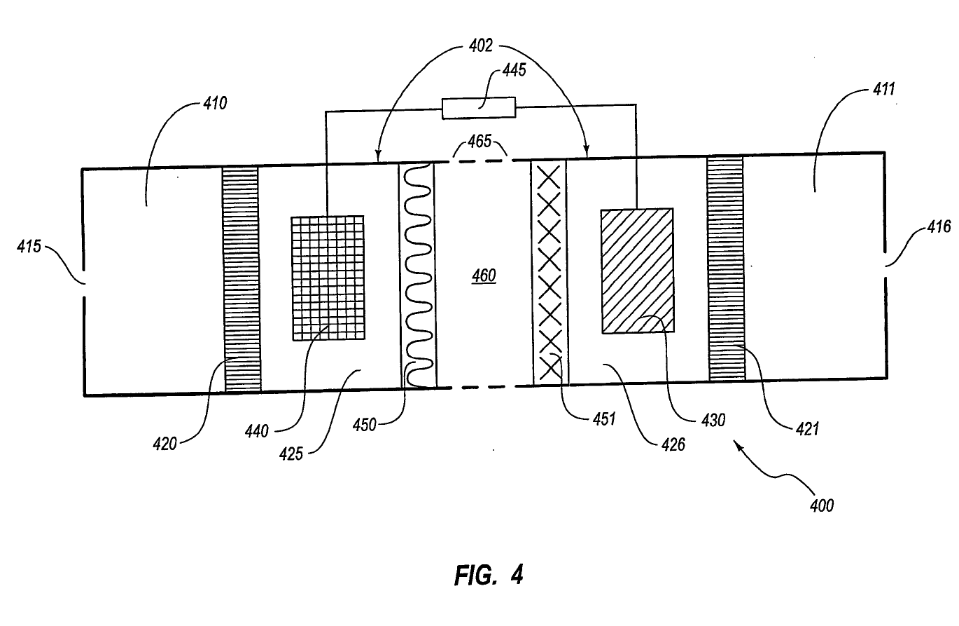

As with the embodiment disclosed in connection with FIG. 3, fluid delivery device 400 of FIG. 4 may also provide a method of decreasing the ratio of the electrochemical engine volume to volume of fluid to be dispensed. FIG. 4 is another embodiment of a dual membrane fluid delivery device 400, which includes an electrochemical pump 402, which may be an electro-osmotic pump comprising a first electrode 430, such as a cathode, coupled to a second electrode 440, such as an anode, via circuit element 445.

The dual membrane fluid delivery device 400 also combines both ANEK and CATEK systems. Anode 440 may be disposed inside first driving chamber 425 and adjacent to an anion exchange membrane 450 and first displaceable member 420, which may be a first piston. Cathode 430 may be disposed inside second driving chamber 426 adjacent a second piston 421 (or alternative displaceable member) and a cation exchange membrane 451.

The fluid delivery device 400 of FIG. 4 also includes a first fluid reservoir 410 for housing a first fluid and a second fluid reservoir 411 for housing a second fluid. First fluid reservoir 410 may be in communication with and receive driving pressure from the first driving chamber 425 and first piston 420, according to the osmotic and electro-osmotic principles described herein. Upon receipt of driving pressure from the first piston 420, first fluid may be dispensed from first port 415. Second fluid reservoir 411 may be in communication with and receive driving pressure from the second driving chamber 426 and second piston 421, according to the osmotic and electro-osmotic principles described herein. Upon receipt of driving pressure from the second piston 421, second fluid may be dispensed from second port 416.

Consequently, the embodiment of FIG. 4 may dispense fluid from two separate reservoirs. In one embodiment, the first fluid and the second fluid are substantially the same, and may comprise a beneficial agent. In an alternative embodiment, the first fluid and the second fluid may be different fluids, such as different beneficial agents that work independently or in concert with each other in a patient. The delivery rate of the first and second fluids can be adjusted by changing the resistance between electrodes 430, 440 when circuit element 445 comprises a resistor, or by creating variable back-pressure through configuration of piston 420, 421 or ports 415, 416.

In embodiments where different fluids are dispensed out of the first and second fluid reservoirs 410, 411, a different volume of fluid may be delivered from the first reservoir 410 compared to the second reservoir 411. For instance, if the diameter of the first fluid reservoir 410 is greater or smaller than the diameter of the second fluid reservoir 411, the volume of first fluid delivered may be different from the volume of second fluid delivered.

The ion and water transport that occurs across the anion 450 and cation 451 exchange membranes may come from body fluid located in aqueous solution chamber 460. Body fluid may enter the aqueous solution chamber 460 of fluid delivery device 400 through orifices 465. Alternatively, a permeable membrane may be utilized instead of orifices 465.

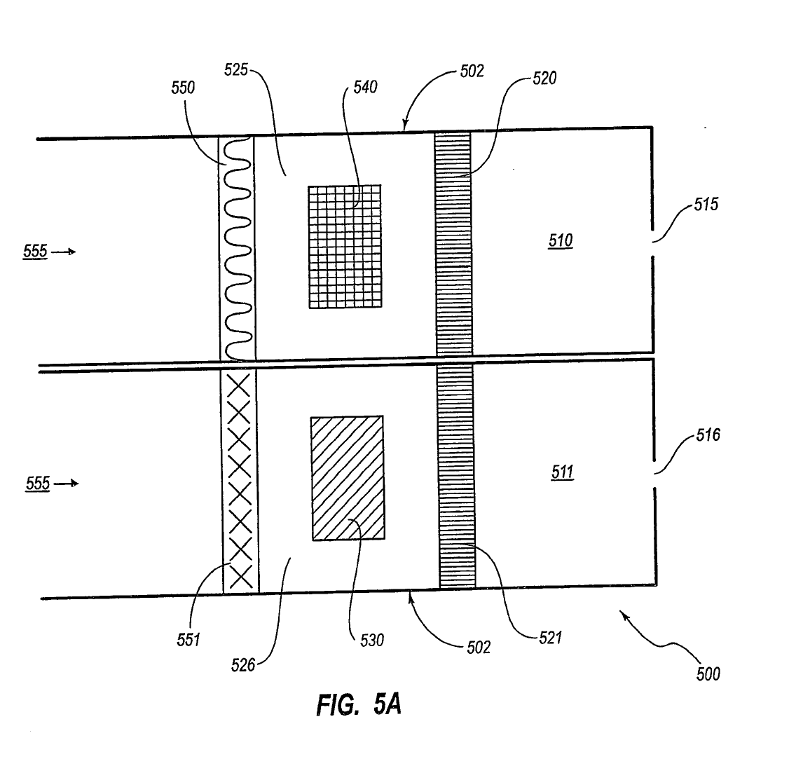

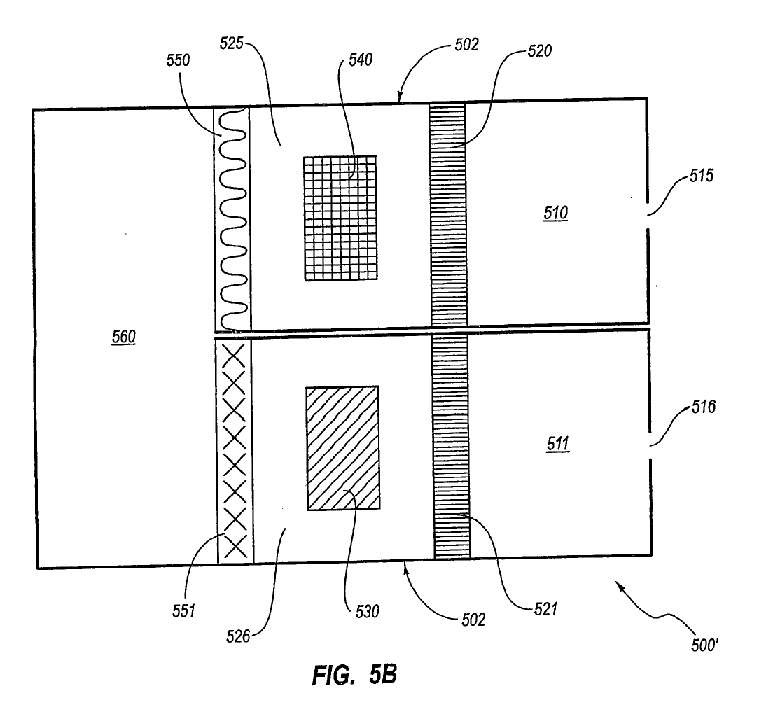

FIG. 5A represents another embodiment of an implantable dual membrane fluid delivery device 500. FIG. 5B represents an embodiment of a dual membrane fluid delivery device 500′ that may be disposed external to a patient. Referring collectively to FIG. 5A and FIG. 5B, fluid delivery devices 500, 500′ include an electrochemical pump 502, which may be an electro-osmotic pump comprising a first electrode 530, such as a cathode, coupled to a second electrode 540, such as an anode, via circuit element (not shown in FIGS. 5A and 5B).

Fluid delivery devices 500, 500′ also combine both ANEK and CATEK systems. Anode 540 may be disposed inside first driving chamber 525 adjacent to anion exchange membrane 550 and first piston 520 (or alternative displaceable member). Cathode 530 may be disposed inside second driving chamber 526 adjacent second piston 521 (or alternative displaceable member) and a cation exchange membrane 551.

Fluid delivery devices 500, 500′ also include a first fluid reservoir 510 for housing a first fluid and a second fluid reservoir 511 for housing a second fluid, which may be dispensed from first 515 and second 516 ports, respectively. First 510 and second 511 fluid reservoirs may be in communication with and receive a driving force from first 520 and second 521 pistons, respectively. The driving force may be generated from pressure from first 525 and second 526 driving chambers according to the osmotic and electro-osmotic principles described herein.

The ratio of the electro-osmotic engine volume to the volume of fluid to be dispensed may further be decreased by mechanically coupling the first piston 520 and/or second piston 521 to one or more slave pistons in one or more additional fluid reservoirs. When the first and/or second pistons 520, 521 are displaced by the electro-osmotic pump 502, they may pull or push on one or more slave pistons that are mechanically coupled thereto.

The embodiment of the implantable fluid delivery device 500 of FIG. 5A, may operate through osmotic and electro-osmotic pressure that is derived from ion and water transport from body fluid 555 passing across ion exchange membranes 550, 551. Alternatively, in the embodiment of the fluid delivery device 500′ of FIG. 5B, which may be disposed external to a patient, osmotic and electro-osmotic pressure may be derived from ion and water transport from saline or another acceptable solution disposed in aqueous solution chamber 560. In one embodiment, the aqueous solution chamber 560 is collapsible.

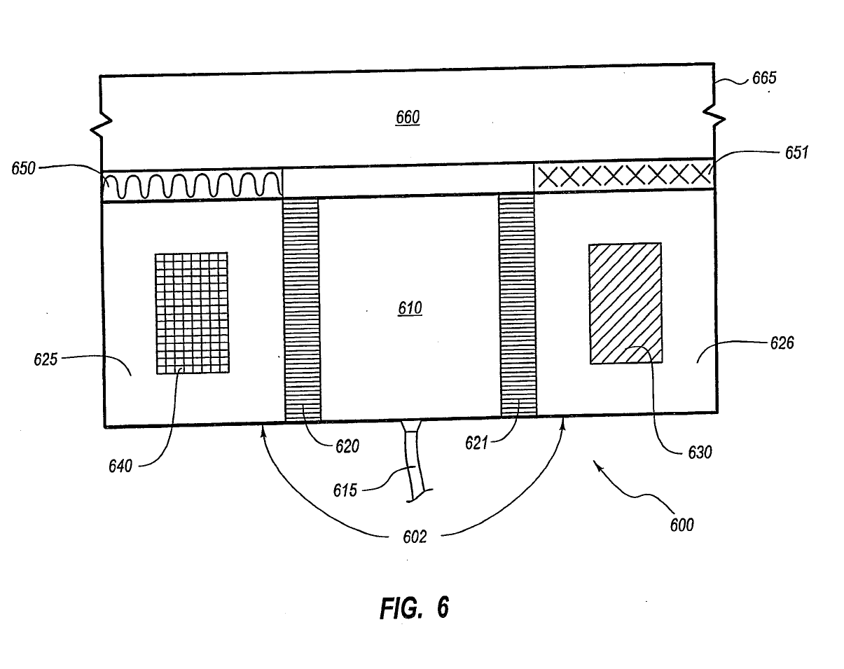

FIG. 6 represents another embodiment of a dual membrane fluid delivery device 600, which may be used external to a patient. Fluid delivery device 600 may include a fluid reservoir 610 to house a fluid such as a beneficial agent, which may be dispensed from a port or catheter 615 or other fluid delivery component. Fluid delivery device 600 also includes an electrochemical pump 602, which may be an electro-osmotic pump comprising a cathode 630 coupled to an anode 640, via circuit element (not shown in FIG. 6).

The dual membrane fluid delivery device 600 also combines both ANEK and CATEK systems. Anode 640 may be disposed inside first driving chamber 625 and adjacent to an anion exchange membrane 650 and first displaceable member 620, which may be a first piston. Cathode 630 may be disposed inside second driving chamber 626 adjacent a second piston 621 (or alternative displaceable member) and a cation exchange membrane 651.

Fluid delivery device 600, which may be disposed external to a patient, may include an aqueous solution chamber 660. Aqueous solution chamber 660 may house saline or another acceptable solution to provide the water and ions that are transported across ion exchange membranes 650, 651 providing osmotic and electro-osmotic pressure to drive the fluid delivery device 600. The aqueous solution chamber 660 may be defined by collapsible walls 665, which can be collapsed or otherwise compressed when the solution inside aqueous solution chamber 660 is transported across the ion exchange membranes 650, 651. This embodiment provides for a smaller overall volume of the fluid delivery device 600 as electro-osmotic and osmotic transport occurs.

Although several particular embodiments, compositions and materials have been disclosed herein, it should be understood that numerous variations thereof are possible as well. For example, each of the fluid reservoirs, bags, bellows, etc., disclosed and described herein can be considered means for housing a fluid. Likewise, each of the pistons, plungers, diaphragms, bladders and bellows described herein, can be considered means for driving the fluid from the delivery device. Furthermore, the electrochemical devices, pumps and engines disclosed herein are examples of means for applying pressure to the driving means.

Without further elaboration, it is believed that one skilled in the art can use the preceding description to utilize the present disclosure to its fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles of the disclosure described herein. In other words, various modifications and improvements of the embodiments specifically disclosed in the description above are within the scope of the appended claims. Note that elements recited in means-plus-function format are intended to be construed in accordance with 35 U.S.C. §112 ¶6. The scope of the invention is therefore defined by the following claims.

1. A fluid delivery device, comprising:
a fluid reservoir configured to contain a fluid to be dispensed; and
an electrochemical pump capable of applying pressure to the fluid reservoir to dispense the fluid, the electrochemical pump, comprising:
a first electrode;
a second electrode;
an anion exchange membrane; and
a cation exchange membrane.

2. The fluid delivery device of claim 1, wherein the electrochemical pump is an electro-osmotic pump comprising a driving chamber capable of retaining water transported across at least one of the membranes into the driving chamber.

3. The fluid delivery device of claim 2, wherein the driving chamber displaces a displaceable member upon transportation of water across the at least one membrane, such that the displaceable member applies pressure to the fluid reservoir to dispense the fluid.

4. The fluid delivery device of claim 3, further comprising:
a second driving chamber and a second displaceable member, wherein the displaceable members comprise first and second pistons, such that first and second pistons simultaneously apply pressure to the fluid reservoir to dispense the fluid when water is transported into each driving chamber.

5. The fluid delivery device of claim 3, further comprising:
a second driving chamber, a second displaceable member and a second fluid reservoir, wherein the displaceable members comprise first and second pistons, such that first and second pistons apply pressure to the fluid reservoirs to dispense the fluid when water is transported into each driving chamber.

6. The fluid delivery device of claim 5, wherein the fluid comprises a first fluid and a second fluid and each fluid reservoir contains a different fluid to be delivered.

7. The fluid delivery device of claim 1, wherein the fluid comprises a beneficial agent.

8. The fluid delivery device of claim 1, wherein the first electrode comprises an anode and the second electrode comprises a cathode, such that the cation exchange membrane is located adjacent the cathode and the anion exchange membrane is located adjacent the anode.

9. The fluid delivery device of claim 8, wherein the anode comprises a zinc anode and the cathode comprises a silver chloride cathode.

10. The fluid delivery device of claim 1, further comprising a resistor coupled between the first electrode and the second electrode.

11. An implantable device for dispensing a beneficial agent, comprising:
a first fluid reservoir having at least one dispensing port, the first fluid reservoir configured to contain a first beneficial agent; and
an electrochemical pump to actuate the dispensing of the first beneficial agent from the first fluid reservoir, the electrochemical pump, comprising:
a first driving chamber comprising a first electrode and an anion exchange membrane; and
a second driving chamber comprising a second electrode and a cation exchange membrane.

12. The device of claim 11, wherein the anion exchange membrane and the cation exchange membrane are exposed to body fluid.

13. The device of claim 11, wherein the electrochemical pump is an electro-osmotic pump capable of transporting water across the anion exchange membrane and the cation exchange membrane into the first driving chamber and the second driving chamber, respectively.

14. The device of claim 11, wherein the at least one dispensing port is coupled to a catheter.

15. The device of claim 11, further comprising:
a second fluid reservoir having at least one dispensing port, the second fluid reservoir configured to contain a second beneficial agent,
wherein the first driving chamber is configured to apply pressure to the first fluid reservoir to dispense the first beneficial agent out of the at least one dispensing port of the first fluid reservoir and the second driving chamber is configured to apply pressure to the second fluid reservoir to dispense the second beneficial agent out of the at least one dispensing port of the second fluid reservoir.

16. The device of claim 15, wherein the first beneficial agent and the second beneficial agent are the same beneficial agent.

17. The device of claim 11, wherein the anion exchange membrane is configured to allow the transport of Cl⁻ across the anion exchange membrane and the cation exchange membrane is configured to allow the transport of Na⁺ across the cation exchange membrane.

18. The device of claim 17, wherein the transport of Cl⁻ and Na⁺ across the anion and cation exchange membranes, respectively, further comprises the transport of a sheath of water molecules along with the transport of Cl⁻ and Na⁺ ions.

19. A fluid delivery device, comprising:
first means for driving a fluid from the delivery device;
second means for driving the fluid from the delivery device; and
means for applying pressure to the first and second driving means, wherein the means for applying pressure comprises an anion exchange membrane and a cation exchange membrane.

20. The fluid delivery device of claim 19, further comprising a first means for housing the fluid, such that the first and second driving means drive the fluid out of the housing means upon receipt of pressure from the means for applying pressure.

21. The fluid delivery device of claim 19, further comprising a first means for housing the fluid, the fluid comprising a first fluid, and further comprising a second means for housing a second fluid, such that the first and second driving means drive the first and second fluids from the first and second housing means, respectively, upon receipt of pressure from the means for applying pressure.

22. The fluid delivery device of claim 21, wherein the first fluid is substantially identical to the second fluid.

23. The fluid delivery device of claim 19, wherein the means for applying pressure comprises an electrochemical pump having a first electrode and a second electrode, the first electrode being disposed in a first driving chamber adjacent the anion exchange membrane and the first driving means and the second electrode being disposed in a second driving chamber adjacent the cation exchange membrane and the second driving means.
